(19) United States
(12) Patent Application Publication
(10) Pub. No.: US 2024/0023647 A1
(43) Pub. Date: Jan. 25, 2024

(54) SMART SHIRTS FOR ASSISTING LUMBAR SPINE AND ITS CONTROL METHOD

(71) Applicant: SEOUL NATIONAL UNIVERSITY R&DB FOUNDATION, Seoul (KR)

(72) Inventors: Sun Gun CHUNG, Seoul (KR); Keewon KIM, Seoul (KR); Ju Chan KIM, Seoul (KR)

(73) Assignee: SEOUL NATIONAL UNIVERSITY R&DB FOUNDATION, Seoul (KR)

(21) Appl. No.: 18/477,000

(22) Filed: Sep. 28, 2023

Related U.S. Application Data
(63) Continuation of application No. PCT/KR2021/009760, filed on Jul. 28, 2021.

(30) Foreign Application Priority Data
Mar. 29, 2021 (KR) 10-2021-0040222

(51) Int. Cl. A41D 13/05; A61B 5/11; A61B 5/00

(52) U.S. Cl. CPC A41D 13/0531; A61B 5/1116; A61B 5/6805; A41D 2200/10

(57) ABSTRACT

Smart shirts for assisting lumbar spine according to an embodiment of the present disclosure includes a clothing body worn by a user; a sensor unit that includes a first sensor and a second sensor provided on a rear surface of the clothing body and disposed in a thoracic portion and a sacral portion, respectively, to measure a motion of the lumbar spine; a control unit that analyzes an operation state of the lumbar spine based on a measurement value of the sensor unit; and a band portion that is worn on the clothing body and surrounds the waist.

### CROSS-REFERENCE TO RELATED APPLICATION

This application is a continuation of International Application No. PCT/KR2021/009760 filed on Jul. 28, 2021, which claims the benefit under 35 USC 119(a) of Korean Patent Application No. 10-2021-0040222 filed on Mar. 29, 2021, in the Korean Intellectual Property Office, the entire disclosures of which are incorporated herein by reference for all purposes.

### BACKGROUND

### 1. Field

The present disclosure relates to smart shirts for assisting lumbar spine. More specifically, the present disclosure relates to smart shirts based on a lumbar spine motion analysis and a variable stiffness lumbar spine assisting device system and a control method thereof.

### 2. Description of the Related Art

70% to 85% of the population experience lumbar spine pain at least once in their lifetime. The annual prevalence of lumbar spine pain is close to 15% to 45%, and the average prevalence at any given time is close to 30%. As such, a very large number of people experience lumbar spine pain. Lumbar spine pain is a disease that not only has a very high lifetime prevalence, but also reduces quality of life due to pain and activity restrictions, and causes great personal and national losses by hindering economic activities. Therefore, preventing and treating lumbar spine pain is a very important task to improve the quality of life of the people and to minimize economic difficulties caused by loss of labor power.

Spinal diseases that cause lumbar spine pain start from damage to the small endplate of the lumbar spine disc or annular tear. In addition, continuous re-injury and incomplete recovery are repeated throughout life, and thereby the disease progresses. Incorrect posture and motion cause damage, which in turn affects posture and motion. Therefore, it is necessary to develop a sensor system that may quantitatively measure posture and motion in daily life and work situations, and to develop a biomechanical index that may analyze the data collected through the sensor system. In particular, due to the mass production and spread of smart phones, various small sensors are being sold at relatively low prices, and mobile platforms are well established. Accordingly, developing a mobile healthcare system using this will greatly contribute to the prevention of spinal diseases.

Clinically, most lumbar spine disc diseases occur at the junction of the lower lumbar spine and sacrolumbar spine and cause damage to the lumbar spine discs. Most of the motions that cause lumbar spine pain include the motion of the junction of the lower lumbar spine and sacrolumbar spine.

The disc between bones of the 4th and 5th lumbar spine and the disc between bones of the 5th lumbar spine and the 1st sacrolumbar spine are the most commonly damaged disc parts. The posture of lifting an object by bending deeply at the waist causes a lot of motion, especially between the lower lumbar spine and the sacrolumbar spine. This posture most often causes damage to the lumbar spine discs. In other words, for the analysis of clinically meaningful lumbar spine motion, it is essential to analyze the motion of the portion corresponding to the sacrolumbar spine in the lower lumbar spine.

Conventionally, there have been spinal motion analysis devices using sensors similar to an attitude heading reference system (AHRS), but it is a difficult situation that clinically meaningful analysis is not possible due to the lack of anatomical and clinical knowledge and the lack of positioning of sensors in appropriate area.

The company of Lumo Bodytech, Inc. invents a device in which a tri-axial accelerometer is attached to the user, the user's posture and motion are measured based on the average of the accelerometer data stored over time, and then feedback is provided with sound or vibration. However, since the lordosis angle of the spine may not be known with only one sensor and only information on the global coordinate may be obtained, it has a limitation in that the relative motion of the spine cannot be known.

The commercialized product of the company of Adela Health Inc. called TruPosture is a device that monitors the posture of the spine with 5 sensors and trains a desired posture with a vibration alarm, and improves usability by inserting the sensor into clothing. Since the product records the posture at five points of the spine, it has significance that it is relatively sophisticated among products on the market. However, there is a limitation in that the motion of the sacrolumbar spine cannot be adequately reflected because the sensor cannot be positioned at an accurate position even though a plurality of sensors are used. In addition, as the number of sensors increases, power consumption increases, which is disadvantageous in use as a wearable device.

In addition to this, conventional products include only feedback such as sounding an alarm based on the analyzed spinal motion, and there is a limitation in that an appropriate operation unit based on this feedback is not included. Lumbar spine orthoses such as belly bands are one of the most commonly prescribed conservative treatments for acute lumbar spine pain, and their effectiveness has been proven. However, due to the difficulty of properly adjusting the intensity, the degree of compliance is low, and when worn with too strong intensity for a long period of time, there is a limit in that atrophy of the muscles around the waist is caused.

In this regard, Korean Patent Registration No. 10-1967665 (Title of Invention: Remote Spine Diagnosis System Using Wearable Measuring Device) discloses a remote spine diagnosis system using a wearable measuring device.

### SUMMARY

Some embodiments of the present disclosure were created to solve the above problems. An object of the present disclosure is to provide a system for evaluating the motion of the lumbar spine using two AHRS sensors, a variable stiffness lumbar spine assisting device system using an electrical adhesive clutch technology controlled based thereon, and smart shirts based thereon.

However, the technical problem to be achieved by the present embodiment is not limited to the technical problems described above, and other technical problems may further exist.

As technical means for achieving the above-described technical problem, smart shirts for assisting lumbar spine according to an embodiment of the present disclosure includes a clothing body worn by a user; a sensor unit that includes a first sensor and a second sensor provided on a rear surface of the clothing body and disposed in a thoracic portion and a sacral portion, respectively, to measure a motion of the lumbar spine; a control unit that analyzes an operation state of the lumbar spine based on a measurement value of the sensor unit; and a band portion that is worn on the clothing body and surrounds the waist.

The clothing body may be formed in a shape of a sleeveless tee, and include a shoulder portion which is configured such that first regions located at positions corresponding to a pair of shoulder blades and a second region located at a position corresponding to the thoracic portion between the first regions; and a tail portion extending from a rear surface of the sleeveless tee and formed to cover the sacral portion.

The clothing body may be formed in a shape of a sleeveless tee, and include a shoulder portion which is configured such that first regions corresponding to a pair of shoulder blades are exposed and a second region located at a position corresponding to the thoracic portion between the first regions covers the thoracic portion.

The shoulder portion may have higher elasticity of the material of the first region than that of the second region.

The band portion may include a bellows type action band; and a rubber band that is located at a lower end of the action band to prevent the tail portion from lifting up according to the user's motion.

The sensor unit may include a first sensor provided in the second region of the shoulder portion to maintain a predetermined position of the thoracic portion; and a second sensor provided on the tail portion to maintain a predetermined position of the sacral portion.

The first sensor and the second sensor may be attitude heading reference system (AHRS) sensors.

The control unit may calculate a biomechanical parameter based on information collected from the first sensor and the second sensor, respectively, analyze the biomechanical parameter, determine an operation state of the lumbar spine, and perform a lumbar spine motion analysis providing feedback to the user based on the determined information.

The control unit may output an analysis value according to the lumbar spine motion analysis, and further include an operation unit that adjusts the stiffness of a belt based on the analysis value of the control unit, the operation unit may be configured of an electrical adhesive clutch including a pair of clutch plates provided on the belt and generating an attractive force attached to each other by an electric signal, and the clutch plate may press the abdomen while tightening the belt or keeping the belt loose according to the presence or absence of an electric signal to adjust the spine stability.

A control method of smart shirts for assisting lumbar spine according to another embodiment of the present disclosure includes a step of collecting data from a sensor unit including a first sensor and a second sensor provided in the smart shirts; a step of calculating a biomechanical parameter based on the collected data and analyzing the biomechanical parameter to analyze a motion state of the lumbar spine; and a step of providing a feedback by outputting an analysis value according to a lumbar spine motion analysis and controlling an operation unit based on the analysis values, in which the smart shirts includes a clothing body worn by a user and a band portion that is worn on the clothing body and surrounds the waist, and the sensor unit includes the first sensor and the second sensor provided on a rear surface of the clothing body and disposed in a thoracic portion and a sacral portion, respectively, to measure a motion of the lumbar spine.

The operation unit may adjust the stiffness of a belt based on the analysis value and be configured of an electrical adhesive clutch including a pair of clutch plates provided on the belt and generating an attractive force attached to each other by an electrical signal, and the clutch plate may adjust spinal stability while pressing the abdomen by tightening the belt or keeping the belt loose depending on the presence or absence of the electrical signal.

According to any one of the above-described problem solving means of the present disclosure, the lumbar spine motion analysis system including two AHRS sensors and appropriate positions according to an embodiment of the present disclosure enables accurate analysis of lumbar spine health and effects of rehabilitation treatment by providing quantitative index for lumbar spine motions that are clinically important in the most efficient manner.

In addition, it is possible to provide more clinically important information than that in the existing systems using one sensor. In addition, compared to the existing systems using three or more sensors, it is possible to provide important clinically necessary information more efficiently.

This lumbar spine motion analysis system may effectively reduce errors in measurement values, which are the core of the system, while increasing usability. In addition, unlike watches and belts, this system is applied to “shirts” that everyone uses by default, and in the process, it is possible to overcome various obstacles that may cause inaccurate measurement values. This has the effect of being able to provide accurate information while being easily usable when the system is developed into an actual product.

In addition, by providing the required strength for an appropriate period through the variable stiffness lumbar spine assisting device of the operation unit, it is possible to increase the compliance of patients with lumbar spine pain and prevent complications such as atrophy of muscles around the lumbar spine that occur when the device is worn for a long period of time.

In addition, if the electrical adhesive clutch technology is used, it is possible to manufacture the assisting device that is lightweight and consumes less power while changing the strength to a desired level.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B are views illustrating smart shirts according to an embodiment of the present disclosure;

FIG. 2A is an enlarged view of a tail portion of the rear surface of the smart shirts illustrated in FIG. 1B;

FIG. 2B is a view illustrating smart shirts manufactured in a shape that does not cover the shoulder blades according to an embodiment of the present disclosure;

FIG. 3 is a view illustrating an operation unit using a sensor unit and an electrical adhesive clutch technology according to an embodiment of the present disclosure;

FIG. 4 is a view for explaining positions of two AHRS sensors according to an embodiment of the present disclosure;

FIGS. 5A, 5B, and 5C are an example of experimental data illustrating changes in sensor measurement values while repeating a motion of picking up an object from the floor after locating the sensor at different positions;

FIG. 6 is a flowchart for explaining the lumbar spine motion analysis and the operation of a variable stiffness lumbar spine assisting device based thereon according to an embodiment of the present disclosure; and

FIG. 7 is a flowchart for explaining a control method of smart shirts according to an embodiment of the present disclosure.

### DETAILED DESCRIPTION

Hereinafter, embodiments of the present disclosure will be described in detail so that those skilled in the art may easily practice them with reference to the accompanying drawings. However, the present disclosure may be embodied in many different forms and is not limited to the embodiments described herein. And in order to clearly explain the present disclosure in the drawings, parts irrelevant to the description are omitted, and similar reference numerals are attached to similar parts throughout the specification.

Throughout the specification, when a part is said to be “connected” to another part, this includes not only the case of being “directly connected”, but also the case of being “electrically connected” with another element therebetween. In addition, when a part “includes” a certain component, this means that it may further include other components, not excluding other components, unless otherwise stated. It should be understood that the presence or addition of one or more other features, numbers, steps, operations, components, parts, or combinations thereof is not precluded.

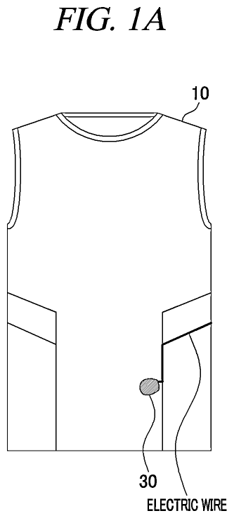

FIGS. 1A and 1B are views illustrating smart shirts according to an embodiment of the present disclosure.

FIG. 1A illustrates a front surface of the smart shirts of the present disclosure and FIG. 1B illustrates a rear surface of the smart shirts.

As illustrated in FIGS. 1A and 1B, the smart shirts based on the lumbar spine motion analysis and variable stiffness lumbar spine assisting device system according to an embodiment of the present disclosure includes a clothing body 10 worn by a user, a sensor unit 20 provided on a rear surface of the clothing body 10 and disposed in the thoracic portion and the sacral portion, respectively, to measure the motion of the lumbar spine, a control unit 30 that analyzes a lumbar spine state based on a measurement value of the sensor unit 20, and a band portion 40 that is worn on the clothing body 10 and surrounds the waist.

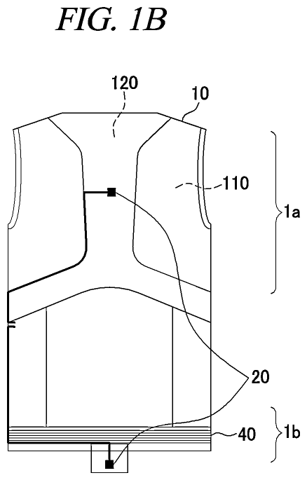

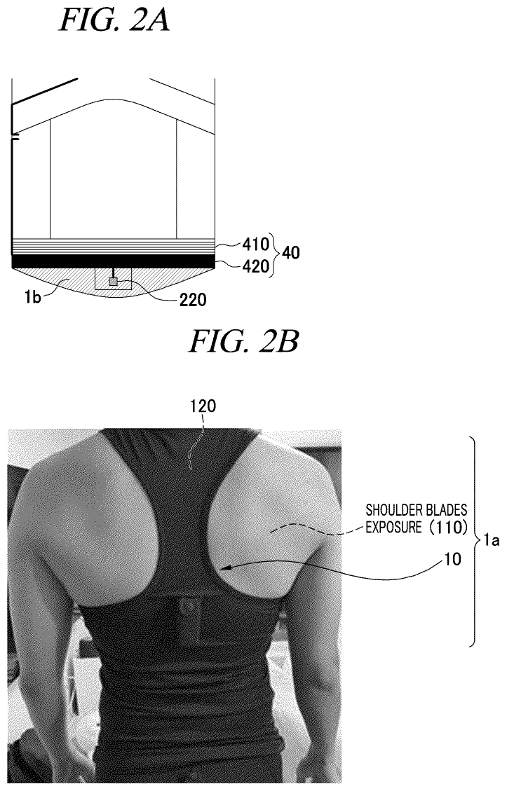

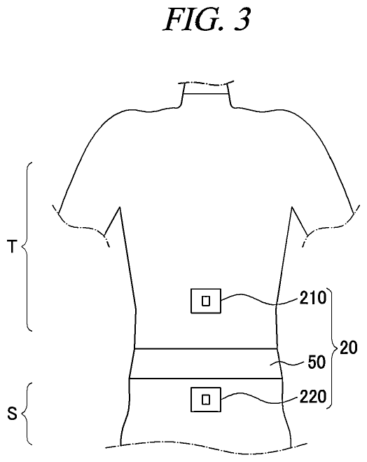

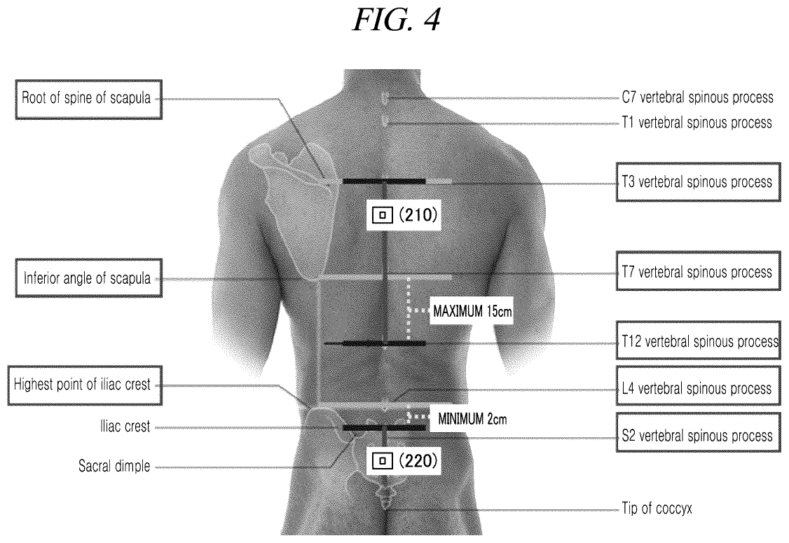

FIG. 2A is an enlarged view of a tail portion of the rear surface of the smart shirts illustrated in FIG. 1B. FIG. 2B is a view illustrating the smart shirts manufactured in a shape that does not cover the shoulder blades according to an embodiment of the present disclosure. FIG. 3 is a view illustrating an operation unit using the sensor unit and an electrical adhesive clutch technology according to an embodiment of the present disclosure. FIG. 4 is a view for explaining positions of two AHRS sensors according to an embodiment of the present disclosure.

As illustrated in FIGS. 1B, 2A, and 3, the sensor unit 20 includes a first sensor 210 provided in a second region 120 of the shoulder portion 1a to maintain a predetermined position of the thoracic portion T and a second sensor 220 provided on the tail portion 1b to maintain a predetermined position of the sacral portion S.

The clothing body 10 is formed in the shape of a sleeveless tee, and includes a shoulder portion 1a configured of first regions 110 where a pair of shoulder blades are located and a second region 120 where the thoracic portion is located between the first regions 110, and a tail portion 1b extending from the rear surface of the sleeveless tee and formed to cover the sacral portion.

Illustratively, the lumbar spine motion analysis system is a system that analyzes the lumbar spine motion using two attitude heading reference system (AHRS) sensors of the thoracic portion T and the sacral portion S, and includes the control unit 30 and the sensor unit 20 provided at appropriate positions. That is, the first sensor 210 and the second sensor 220 may be configured as the AHRS sensors.

In the lumbar spine motion analysis system, it is important to precisely locate the position of the sensor unit 20, and it is particularly important that the position does not change during use by one individual. Accordingly, in the present disclosure, it is important that the sensor unit 20 maintains a predetermined position in order to secure an accurate position of the sensor unit 20 and minimize errors in measurement values while applying the system within one smart shirts. In addition, the placement position of the control unit 30 to improve usability and comfort may also be considered when manufacturing the smart shirts.

Referring to FIGS. 1A and 1B, the shoulder portion 1a is a portion where the first sensor is located, and is a device for minimizing interference received by the first sensor due to motion of the arm and the shoulder blade.

The tail portion 1b is a part where the second sensor is located, and is a device for maintaining the second sensor at a predetermined position and preventing the position from changing even when worn for a long period of time.

In addition, the smart shirts may be made of a fabric formed of a material such as polyester with high elasticity. This is to increase the accuracy of the measurement value by causing the sensor to come into close contact with the body.

Referring to FIG. 2B, the clothing body 10 is formed in the shape of the sleeveless tee, and may include the shoulder portion 1a which is configured such that the first regions 110 corresponding to a pair of shoulder blades are exposed and the second region 120 located at a position corresponding to the thoracic portion between the first regions 110 to cover the thoracic portion. Illustratively, the shoulder portion 1a may be formed such that the first region 110 of the sleeveless tee is removed to expose the shoulder blade. In addition, the shoulder portion 1a may be formed with higher elasticity of the material of the first region 110 than that of the second region 120. That is, the shoulder portion 1a may have a shape that does not cover the shoulder blade portion, or may be manufactured by using different materials for the portion of the shoulder blades and the portion between the shoulder blade portions.

When the material of the portion of the shoulder blades and the portion between the shoulder blade portions are different when manufacturing the shoulder portion 1a, a material with high elasticity may be used for the portion of the shoulder blades, and a hard material with low elasticity may be used for the portion between the shoulder blades.

It is not difficult to locate the first sensor 210 at a desired position, and there is little risk of its position changing significantly during use. However, when the first sensor 210 is located on normal shirts, the value of the first sensor 210 greatly changes according to the motion of the arm. For example, when the sensor was located on general-shaped T-shirt and flexion was performed to the maximum range of motion of the shoulder joint, an error of about 10 degrees occurred in addition to the physiological spine motion. However, when the smart shirts is designed in the shape of the sleeveless tee so as not to cover the first region 110 of the shoulder portion 1a according to an embodiment of the present disclosure, the sensor is located, and the same motion is performed, it was confirmed that no error almost occurred In this way, the present disclosure may reduce the change in the value of the sensor by the motion of the arm and the shoulder blade through the shoulder portion 1a of the above-described smart shirts.

The control unit 30 is provided on the front surface of the clothing body 10, and includes a microprocessor, a battery for supplying power, and a wireless communication unit.

Illustratively, as illustrated in FIG. 1A, the control unit 30 may be configured of a box-shaped device including a microprocessor, a battery, and a wireless communication unit, and is attached to the front surface of the smart shirt and may be located on a side surface of the lower abdominal portion or a lower portion of the clavicle. In addition, the control unit 30 and the sensor unit 20 may be connected by wires located along a sewing line. Accordingly, when the user wears the smart shirts, it is possible to increase the wearing comfort. The wireless communication unit provides a wireless communication interface such as Wifi or Bluetooth, through which the control unit 30 may transmit various types of data generated in the process of wearing the smart shirts to an external smart terminal via wireless communication with various smart terminals. The smart terminal may provide, via a user application, guidance on wearing the smart shirts, various data collected through the first and second sensors, biomechanical parameters, information on the operating state of the operation unit, and the like.

The sensor unit 20 must be located on the back side for accurate measurement, and is configured of two AHRS sensors. At this time, the product that mainly occupies a volume is the battery. Therefore, as described above, the present disclosure separates the battery, places only the AHRS sensor on the back side (rear side) of the smart shirts, attaches it to the clothing body 10, and may locate, on a side (front surface) which is the less uncomfortable side, various products including the battery occupying a large volume to cause major discomfort. Through this, almost no foreign matter sensation is felt on the back side, and it is possible to reduce the risk of greatly inaccurate measurement values when the first and second sensors 210 and 220 come into contact with each other. That is, the smart shirts of the present disclosure is an invention that affects both the improvement of wearing comfort and the minimization of errors in measurement values.

In addition, the smart shirts of the present disclosure includes the band portion 40 that is located at the exact position where the sensor unit 20 is required and does not change during use, and is minimally affected by motions other than that of the spine.

The band portion 40 includes a bellows type action band 410 and a rubber band 420 that is located at a lower end of the action band 410 to prevent the tail portion 1b from lifting up according to the user's motion.

Illustratively, the rubber band 420 may be located on the L4 vertebral spinous process of the 4th lumbar spine. It belongs to a part where the waist is the narrowest in the general body structure. Therefore, when the rubber band 420 is located at this position, its position hardly changes even when worn for a long period of time.

In addition, the action band 410 is a pleated bellows-shaped band. The action band 410 is located at an upper end of the rubber band 420. This serves to prevent the rubber band 420 below from being lifted up by extending only the bellows band while the pleat portion is stretched when the waist is deeply bent.

The tail portion 1b is a part of the smart shirts that is additionally connected to the lower end of the rubber band 420. At this time, the second sensor 220 may be provided on the tail portion 1b to maintain a predetermined position. The second sensor 220 may be located at a position least 2 cm below the rubber band 420 according to the above-described lumbar spine motion analysis system. That is, as the position of the rubber band 420 is constant even when worn for a long period of time, the position of the second sensor 220 hardly changes.

Smart shirts to which the tail portion 1b and the band portion 40 are applied according to an embodiment of the present disclosure was manufactured, and the effect was actually confirmed. As a result, it was confirmed that only the portion of the action band 410 was stretched by the pleat thereof, and accordingly, it was confirmed that the positions of the rubber band 420 and the second sensor 220 below it were maintained.

In an actual long-term wearing experiment, when the tail portion 1b and the band portion 40 were not applied, the position of the second sensor 220 changed by more than 3 cm, whereas when the tail portion 1b and the band portion 40 were applied. it was confirmed that the change in position of the second sensor 220 was less than 1 cm.

In this way, in the case of the second sensor 220, the second sensor 220 should be located at the exact position of the pelvis portion according to the lumbar spine motion analysis system, but when wearing general clothes for a long period of time and repeating sitting or standing, as clothes lifts above the pelvis, the position changes. Therefore, the present disclosure, in order to solve this problem, the second sensor 220 is located at the exact position required by the lumbar spine motion analysis system, and in order to cause the position to be not changed, the above-described tail portion 1b and the band portion are provided.

Referring to FIGS. 3 and 4, it is important that the first and second sensors 210 and 220 are located at the exact positions in order to appropriately reflect the motions of the thoracic and sacrolumbar, respectively. For example, the first sensor 210 of the thoracic portion T may be located between the spine of scapula on both sides and a point cm below the T7 vertebral spinous process, and the second sensor 220 of the sacral portion S may be located at a point at least 2 cm below the L4 vertebral spinous process of the fourth lumbar spine.

Specifically, the AHRS sensors may be attached to the thoracic portion T and the sacral portion S, respectively, and may measure the three-dimensional posture and motion of the spine at each location. Illustratively, the first sensor 210 may be attached between the spine of scapula on both sides and a point 15 cm below the T7 vertebral spinous process.

More precisely, the first sensor 210 should be located between the T3 vertebral spinous process and the T12 vertebral spinous process. For example, the position of the T3 vertebral spinous process is confirmed by palpating the position of the spine of the scapula on both sides and palpating the central portion of the spine at that height. In addition, the position of the T12 vertebral spinous process is confirmed as follows. If the inferior angle of the scapula on both sides is palpated and the spinous process in the center portion of the spine at that height, this is the T7 vertebral spinous process. The T12 vertebral spinous process may be confirmed by palpating the 5 spinous processes below T12 vertebral spinous process, which may be located up to 15 cm below.

Therefore, the first sensor 210 may be attached between the spine of the scapula on both sides and a point 15 cm below the T7 vertebral spinous process.

In addition, the second sensor 220 may be located below the S1 vertebral spinous process. For example, the position of the S1 vertebral spinous process is confirmed as follows. If the highest point of the iliac crest is palpated and the central portion of the spine at that height is palpated, this is the L4 vertebral spinous process. The S1 vertebral spinous process may be confirmed by palpating the two spinous processes below it, which may be located at least 2 cm below.

In other words, for clinically meaningful motion analysis of the lumbar spine, the motion between the lower lumbar spine and the sacrolumbar must be included, and to do this, you need to position the sensor must be located above and below it. Accordingly, two sensors are essential, and it is essential to locate the first and second sensors 210 and 220 respectively at the upper thoracic portion T of the lumbar spine and the lower sacral portion S of the lumbar spine.

Referring to FIG. 3, the smart shirts according to an embodiment of the present disclosure further includes the operation unit 50 that adjusts the stiffness of the belt based on the analysis value of the control unit 30.

Illustratively, the operation unit 50 is configured of an electrical adhesive clutch including a pair of clutch plates provided on the belt and generating an attractive force attached to each other by an electrical signal, and the clutch plate may adjust spinal stability while pressing the abdomen by tightening the belt or keeping the belt loose depending on the presence or absence of an electrical signal.

As an example, the clutch plates are disposed at both ends of the belt, and when an electrical signal is generated, the clutch plates are attached to increase the stiffness of the belt and press the abdomen. As another example, the clutch plates are disposed between two layers of the belt, and when an electric signal is generated, the clutch plates are attached to increase the stiffness of the belt and press the abdomen. On the other hand, when the electrical signal is removed, the attractive force on the clutch plates is removed, allowing the belt to keep loose. On the other hand, the clutch plate is configured of a carbon fiber bar formed of a flexible material, an electrode plate, and an insulator, and the electrical adhesive clutch including this uses a conventionally known technology, and thus a detailed description thereof will be omitted.

For example, the variable stiffness lumbar spine assisting device system refers to the operation unit 50, and is a variable stiffness lumbar spine assisting device using electrical adhesive clutch technology to assist the lumbar spine with appropriate strength. The health of the lumbar spine based on the data measured using the lumbar spine motion analysis system may be evaluated, and then appropriate strength may be set and provided based thereon.

For example, the operation unit 50 may be manufactured to surround the waist of the user equipped with the lumbar spine motion analysis system in a cylindrical shape. The operation unit 50 may increase the abdominal pressure of the user with its own stiffness and help prevent a proper waist posture from collapsing when the user bends or straightens the waist. The operation unit 50 may be provided to generate an appropriate level of stiffness based on the analysis value of the biomechanical parameter. The change in stiffness of the operation unit 50 may be implemented through the above-described electrical adhesive clutch technology.

As an example, the electrical adhesive clutch technology used to change the strength of the lumbar spine assisting device to a desired degree is a technology that is used to improve the function of a spring or actuator in a robot device, and may use less power, be light, and change stiffness. Stuart Diller, 2011, is based on electrostatic adhesion between thin electrode sheets coated with a dielectric material, and various levels of stiffness may be created by disposing multiple clutch springs in parallel to individually adjust the stiffness.

The variable stiffness lumbar spine assisting device system is configured of a belt-type lumbar spine support to which the electrical adhesive clutch technology is applied. The variable stiffness lumbar spine assisting device system is manufactured to surround the user's waist in a cylindrical shape, and may help to increase the user's abdominal pressure with its own stiffness and prevent the user from collapsing in an appropriate waist posture when the user bends or straightens the waist. The stiffness may be adjusted through the electrical adhesive clutch technology based on the health of the lumbar spine.

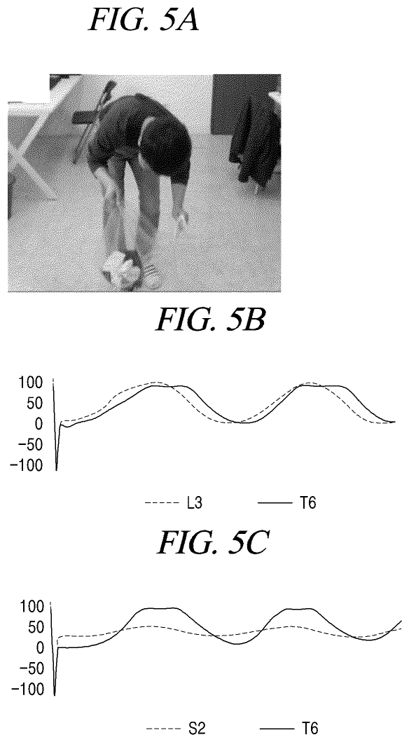

FIGS. 5A, 5B, and 5C are an example of experimental data illustrating changes in sensor measurement values while repeating a motion of picking up an object from the floor after locating the sensor at different positions.

FIGS. 5B and 5C are an example of experimental data of the lumbar spine motion analysis system, which confirms the change in measurement values according to the sensor position while picking up the object from the floor (see FIG. 5A), which is a representative motion that induces lumbar spine pain. After locating the first sensor 210 on the T6 vertebral spinous process, the second sensor 220 is located on the L3 vertebral spinous process or the S2 vertebral spinous process (see FIG. 5C), and thereby the motion is performed. The values indicated in the graphs illustrated in FIGS. 5B and 5C are obtained by quantifying the angle of the sagittal plane of the lumbar spine by measuring the roll value from the AHRS sensor.

When the second sensor 220 is located on the L3 vertebral spinous process while repeatedly performing the motion illustrated in FIG. 5A (see FIG. 5B), it may be confirmed that the value while the motion progresses moves almost identically to that of the first sensor 210. On the other hand, when the second sensor 220 is located on the S2 vertebral spinous process (see FIG. 5C), it may be confirmed that the difference in values is large when the motion is sufficiently advanced. Therefore, it could be observed that the measurement value was more clearly confirmed through the sensor when the second sensor 220 of the sacral portion was located on the S2 vertebral spinous process.

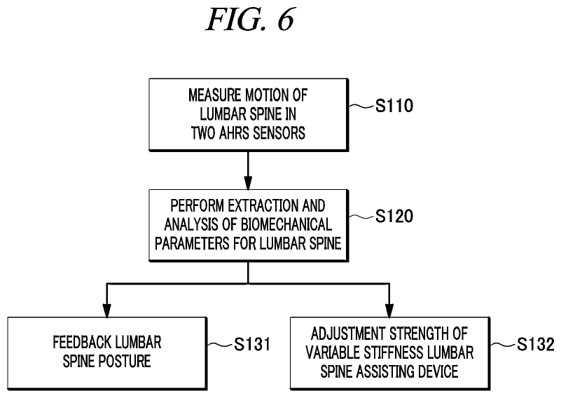

FIG. 6 is a flowchart for explaining the lumbar spine motion analysis and the operation of a variable stiffness lumbar spine assisting device based thereon according to an embodiment of the present disclosure.

Referring to FIG. 6, according to an embodiment of the present disclosure, in the smart shirts, the motion of the lumbar spine may be measured in two AHRS sensor units (S110), extraction and analysis of the biomechanical parameters for the lumbar spine may be performed in the control unit 30 (S120), the lumbar spine posture feedback may be provided to the feedback unit according to the information determined using the biomechanical parameters by the control unit 30 (S131), or the strength of the variable stiffness lumbar spine assisting device (operation unit) may be be adjusted (S132).

For example, the lumbar spine motion analysis system refers to the control unit 30, extracts and analyzes the biomechanical parameters based on data obtained from the sensor unit 20 to analyze the state of the lumbar spine of the subject, and evaluates the state of the lumbar spine to provide feedback or play a role of controlling the strength of the operation unit.

Here, the biomechanical parameters include all parameter combinations in the aforementioned sensors. That is, the state of the lumbar spine is evaluated through mathematical calculation and combinations of data measured by two sensors. For example, a combination of parameters within the sensors may be a combination of the relative angle of the spine and a stability index. Through this, if the posture, stability, and state index of the spine are impaired, feedback is provided or the strength of the variable stiffness lumbar spine assisting device (operation unit) is adjusted.

In addition, the biomechanical parameters may include mean lordosis angle, minimum lordosis angle, maximum lordosis angle, initial lordosis angle, mean spine lateral bending, minimum spine lateral bending, maximum spine lateral bending, initial spine lateral bending, mean spine axial twist, minimum spine axial twist, maximum spine axial twist, initial spine axial twist, square mean spine lordosis angular velocity, mean bending angular velocity, maximum bending angular velocity, mean extension angular velocity, maximum extension angular velocity, square mean spine lateral bending angular velocity, mean spine lateral bending angular velocity, maximum lateral bending angular velocity, square mean spinal axial twist angular velocity, mean spinal axial twist angular velocity, maximum axial twist angular velocity, square mean lordosis angular acceleration, mean bending angular acceleration, maximum bending angular acceleration, mean extension angular acceleration, maximum extension angular acceleration, range of motion, phase-plot area, stability index, jerk index square mean spine lateral bending angular acceleration, mean spine lateral bending angular acceleration, maximum spine lateral bending angular acceleration, range of motion in the pitch direction, phase-plot area in the pitch direction, square mean spine axial twist angular acceleration, mean spinal axial twist angular acceleration, maximum spinal axial twist angular acceleration, range of motion in the yaw direction, phase-plot area in the yaw direction, and a combination of two or more of these.

At this time, primary data obtained through the AHRS sensor unit 20 is defined as a first parameter to analyze the lumbar spine state of the subject.

For example, the first parameter may be an mean lordosis angle, which is an mean of differences in roll direction angles measured by the first sensor and the second sensor, respectively.

The control unit 30 may derive a second parameter by combining a plurality of first parameters, and the control unit 30 may analyze the second parameter to determine the lumbar spine state.

For example, the second parameter may be derived by combining the plurality of first parameters, and the second parameter may be an index of lordosis robustness. The lordosis robustness index may be derived through the mean lordosis angle and the square mean lordosis angular velocity, which are the first parameters. Specifically, the lordosis robustness index may be a value obtained by dividing the mean lordosis angle by the square mean lordosis angular velocity. The lordosis robustness index is an index indicating how well the lordosis is maintained during a specified time or during exercise of the spine. The unit of the lordosis robustness index is ‘second’, and the higher the lordosis of the spine is during the corresponding time or exercise, the higher the value is. That is, the more stable the lordosis of the spine is maintained, the higher the value of the lordosis robustness index is.

In addition, the second parameter may be an instantaneous lordosis robustness index. The instantaneous lordosis robustness index may be derived through a value obtained by dividing the above-described instantaneous lordosis angle by the instantaneous lordosis angular velocity.

As described above, the control unit 30 may determine the state of the lumbar spine through the first parameter and the second parameter, and the operation unit 50 may receive the information determined by the control unit 30 and adjust the spine stability through the user's abdominal pressure control based on the received information.

In addition, the control unit 30 may provide feedback by transferring information determined through the aforementioned biomechanical parameters to the feedback unit. For example, the feedback unit includes a wearable device, a mobile device, and the like. The feedback unit may transmit a signal to the subject based thereon.

The feedback unit may provide feedback to the user when the analysis value of the biomechanical parameter is not appropriate. The feedback unit may provide feedback to the user when the analysis value of the biomechanical parameter is out of a target range. The feedback unit includes at least one feedback method among sound, vibration, pressure, light, and temperature.

Hereinafter, a description of a configuration performing the same function among the configurations illustrated in FIGS. 1A to 6 will be omitted.

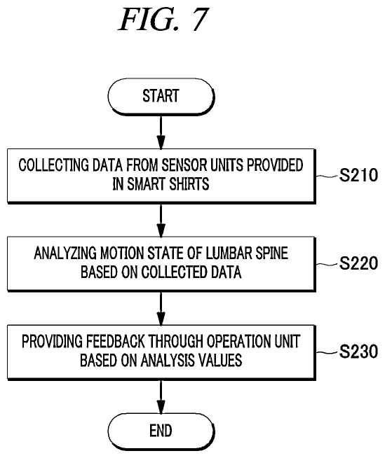

FIG. 7 is a flowchart for explaining a control method of smart shirts according to an embodiment of the present disclosure.

Referring to FIG. 7, a method for analyzing lumbar spine motion performed by the control unit according to an embodiment of the present disclosure includes a step of collecting data from the sensor units (first sensor and second sensor (AHRS)) provided in the smart shirts (S210), a step of calculating biomechanical parameters based on the collected data and analyzing the biomechanical parameters to analyze the motion state of the lumbar spine (S220), and a step of providing a feedback by outputting analysis values according to the lumbar spine motion analysis and controlling the operation unit based on the analysis values (S230).

Illustratively, the control unit may derive the first parameter through any one or more of the measured data, derive the second parameter by combining the plurality of first parameters, and analyze the second parameter to determine the motion state of the lumbar spine, and provide the feedback to the user through the operation unit (variable stiffness lumbar spine assisting device) based on the determined information.

It will be understood that the above description of the present disclosure is for illustrative purposes, and those skilled in the art may easily modify it into other specific forms without changing the technical spirit or essential features of the present disclosure. Therefore, the embodiments described above should be understood as illustrative in all respects and not limiting. For example, each component described as a single type may be implemented in a distributed manner, and similarly, components described as distributed may be implemented in a combined form.

It should be interpreted that the scope of the present disclosure is indicated by the claims to be described later rather than the detailed description above, and all changes or modifications derived from the meaning and scope of the claims and equivalent concepts thereof are included in the scope of the present disclosure.

What is claimed is:

1. A smart shirts for assisting lumbar spine, comprising:
a clothing body worn by a user;
a sensor unit that includes a first sensor and a second sensor provided on a rear surface of the clothing body and disposed in a thoracic portion and a sacral portion, respectively, to measure a motion of the lumbar spine;
a control unit that analyzes an operation state of the lumbar spine based on a measurement value of the sensor unit

2. The smart shirts of claim 1, further comprising a band portion that is worn on the clothing body and surrounds the waist, and
a tail portion extending from a rear surface of the clothing body and formed to cover the sacral portion.

3. The smart shirts of claim 1, wherein the clothing body includes
a shoulder portion which is configured such that first regions corresponding to a pair of shoulder blades are exposed and a second region located at a position corresponding to the thoracic portion between the first regions covers the thoracic portion.

4. The smart shirts of claim 1, wherein the clothing body includes
a shoulder portion which is configured such that first regions located at positions corresponding to a pair of shoulder blades and a second region located at a position corresponding to the thoracic portion between the first regions; and
a tail portion extending from a rear surface of the clothing body and formed to cover the sacral portion.

5. The smart shirts of claim 4, wherein the shoulder portion has higher elasticity of the material of the first region than that of the second region.

6. The smart shirts of claim 4, wherein the band portion includes
a bellows type action band; and
a rubber band that is located at a lower end of the action band to prevent the tail portion from lifting up according to the user's motion.

7. The smart shirts of claim 4, wherein the sensor unit includes
a first sensor provided in the second region of the shoulder portion to maintain a predetermined position of the thoracic portion; and
a second sensor provided on the tail portion to maintain a predetermined position of the sacral portion.

8. The smart shirts of claim 1, wherein the first sensor and the second sensor are attitude heading reference system (AHRS) sensors.

9. The smart shirts of claim 1, wherein the control unit calculates a biomechanical parameter based on information collected from the first sensor and the second sensor, respectively, analyzes the biomechanical parameter, determines an operation state of the lumbar spine, and performs a lumbar spine motion analysis providing feedback to the user based on the determined information.

10. The smart shirts of claim 9, wherein the control unit outputs an analysis value according to the lumbar spine motion analysis, and further includes an operation unit that adjusts the stiffness of a belt based on the analysis value of the control unit,
the operation unit is configured of an electrical adhesive clutch including a pair of clutch plates provided on the belt and generating an attractive force attached to each other by an electric signal, and
the clutch plate presses the abdomen while tightening the belt or keeping the belt loose according to the presence or absence of an electric signal to adjust the spine stability.

11. A control method of smart shirts for assisting lumbar spine, comprising:
collecting data from a sensor unit including a first sensor and a second sensor provided in the smart shirts;
calculating a biomechanical parameter based on the collected data by a control unit and analyzing the biomechanical parameter to analyze a motion state of the lumbar spine; and
providing a feedback by outputting an analysis value according to a lumbar spine motion analysis and controlling an operation unit based on the analysis values by the control unit,
wherein the smart shirts includes a clothing body worn by a user, and
the sensor unit includes the first sensor and the second sensor provided on a rear surface of the clothing body and disposed in a thoracic portion and a sacral portion, respectively, to measure a motion of the lumbar spine.

12. The control method of smart shirts for assisting lumbar spine of claim 11, wherein the smart shirts further includes a band portion that is worn on the clothing body and surrounds the waist.

13. The control method of smart shirts for assisting lumbar spine of claim 11, wherein the operation unit adjusts the stiffness of a belt based on the analysis value, and
is configured of an electrical adhesive clutch including a pair of clutch plates provided on the belt and generating an attractive force attached to each other by an electrical signal, and
the clutch plate adjusts spinal stability while pressing the abdomen by tightening the belt or keeping the belt loose depending on the presence or absence of the electrical signal.
